(19) United States
(12) Patent Application Publication
(10) Pub. No.: US 2018/0000774 A1
(43) Pub. Date: Jan. 4, 2018

(54) METHODS OF TREATING COGNITIVE AND BEHAVIORAL IMPAIRMENT IN DOWN SYNDROME AND ALZHEIMERS DISEASE PATIENTS

(71) Applicant: AVANTI BIOSCIENCES, INC., EAST SETAUKET, NY (US)

(72) Inventor: GIAN LUCA ARALDI, EAST SETAUKET, NY (US)

(73) Assignee: AVANTI BIOSCIENCES, INC, EAST SETAUKET, NY (US)

(21) Appl. No.: 15/634,604

(22) Filed: Jun. 27, 2017

Related U.S. Application Data
(60) Provisional application No. 62/355,976, filed on Jun. 29, 2016.

(51) Int. Cl. A61K 31/353

(52) U.S. Cl. CPC A61K 31/353

(57) ABSTRACT

The present invention relates to methods of treating cognitive and behavioral impairment in Down syndrome and/or Alzheimer's disease patients, Alzheimer's disease, neurodegenerative disease, cancer, DYRK1A-mediated disorders and methods of modulating and inhibiting DYRK1-A comprising use of catechins.

### CROSS-REFERENCE TO RELATED APPLICATIONS

This application claims priority from U.S. Provisional Application No. 62/355,976 filed Jun. 29, 2016 “Methods of Treating Cognitive and Behavioral Impairment in Down Syndrome and Alzheimer's Disease Patients” which is incorporated herein by reference in its entirety.

### GOVERNMENT INTERESTS

Not applicable

### PARTIES TO A JOINT RESEARCH AGREEMENT

Not applicable

### FIELD OF THE INVENTION

The present invention relates methods of treating cognitive and behavioral impairment in Down syndrome and Alzheimer's disease patients, Alzheimer's disease and cancer.

### BACKGROUND OF RELATED TECHNOLOGY

DYRK1A is one of five kinases (DYRK1B, DYRK2, DYRK3 and DYRK4) belonging to the dual-specificity tyrosine phosphorylation-regulated kinase family. The gene encoding DYRK1A is located on chromosome 21 in the Down-Syndrome Critical Region. Because those afflicted with Down Syndrome (DS) have an extra copy of chromosome 21, three copies of the DYRK1A gene are present in DS patients instead of two. This results in 1.5-fold increase of DYRK1A gene expression and DYRK1A activity. R. Abbassi et al., Pharmacology & Therapeutics 151 (2015) 87-98. Studies have shown that DYRK1A over expression induces neuronal progenitors to prematurely differentiate which stunts the formation of mature neurons. Hämmerle, B. et al., Development 138, (2011) 2543-2554. Other studies also demonstrated the neuropathological consequences of increased DYRK1A activity that could manifest the cognitive and behavioral impairments seen in DS patients. Becker, W. et al., CNS Neurol Disord Drug Targets 13, (2014) 26-33; Tejedor, F. J., & Hämmerle, B., FEBS J 278, (2011) 223-235.

Most DS patients are beset with early-onset Alzheimer's disease (AD). AD is characterized by the accumulation of the β-amyloid peptide (Aβ) within the brain through proteolysis of amyloid precursor protein (APP). The APP gene is located on chromosome 21 which is over-expressed in DS patients. Studies have shown that DYRK1A phosphorylates APP which promotes its cleavage by secretases. The resultant peptides (Aβ40 and Aβ42) form the tell-tale amyloid plaques of AD. Wegiel, J., et al., FEBS J 278, (2011) 236-245. The other morphological hallmark of AD are tangles caused by overproduction or phosphorylation of Tau protein. Studies have shown that over expression of DYRK1A hyperphosphorylates Tau and suggest that the extra copy of the DYRK1A gene in DS patients could contribute to their early onset of AD. Ryoo, S. R., et al., J Biol Chem. 2007 Nov. 30; 282(48): 34850-7.

Studies have shown DYRK1A to have both tumor-suppressive and oncogenic properties. DS patients suffer from an increased risk of acute megakaryoblastic leukemia (AMKL) and acute lymphoblastic leukemia (ALL). Studies have shown that DYRK1A promotes megakaryoblastic tumorigenesis by allowing the chromatin of rapidly inducible genes to remain permanently open leading to over-expression of cytokines in DS-associated megakaryoblastic leukaemia. Jang, S. M, et al., EMBO Rep 15, (2014) 686-694. On the other hand, studies have shown that Lewis lung carcinoma and B16F10 melanoma were significantly suppressed in murine Ts65Dn Down syndrome animals compared to controls. R. Abbassi et al., Pharmacology & Therapeutics 151 (2015) 87-98. Anti-cancer activity of DYRK1A is also linked to the inactivation of other oncogenic proteins such as NFAT and cyclin D1. Arron, J. R., et al., Nature 441, (2006) 595-600; Chen, J.-Y., et al., Mol Cell 52, (2013) 87-100.

Epigallocatechin gallate (EGCG) is the primary flavonoid of green tea and has been widely investigated for its therapeutic effects which include anti-inflammatory, anti-oxidative, anti-cancer, anti-infective and neuroprotective activity. Bhat et al. Bioorganic & Medicinal Chemistry Letters 24 (2014) 2263-2266. It is a non-ATP competitive DYRK1A inhibitor (IC50=330 nM). Bain, J., et al., Biochem J 408, (2007) 297-315. In 2014, studies showed that green tea extract comprising 41% EGCG was able to rehabilitate the cognitive decline seen in transgenic mice over expressing DYRK1A. In a randomized, double-blinded placebo-controlled study with DS patients, ECGC also improved memory recognition, working memory and quality of life. De la Torre, R. et al. Mol Nutr Food Res. 2014 February; 58(2):278-88. Thus, there is a need to develop DYRK1A modulators to treat DS, AD and cancer.

### SUMMARY OF THE INVENTION

The present invention is directed to the following exemplary and non-limiting embodiments:

A method of improving cognitive and behavioral impairment in a DS patient in need thereof comprising administering to said patient a therapeutically effective amount of a pharmaceutically acceptable composition comprising a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate, (2R,3S)-2-(3,4-dihydroxyphenyl)-5,7-dihydroxychroman-3-yl 3,4,5-trihydroxybenzoate, (2R,3R)-2-(3,4-dihydroxyphenyl)-5,7-dihydroxychroman-3-yl 3,4,5-trihydroxybenzoate and (2S,3S)-2-(3,4-dihydroxyphenyl)-5,7-dihydroxychroman-3-yl 3,4,5-trihydroxybenzoate or a pharmaceutically acceptable salt thereof.

A method of improving cognitive and behavioral impairment in a DS patient in need thereof comprising administering to said patient a therapeutically effective amount of a pharmaceutically acceptable composition comprising a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate and (2R,3S)-2-(3,4-dihydroxyphenyl)-5,7-dihydroxychroman-3-yl 3,4,5-trihydroxybenzoate or a pharmaceutically acceptable salt thereof.

A method of improving cognitive and behavioral impairment in a DS patient in need thereof comprising administering to said patient a therapeutically effective amount of a pharmaceutically acceptable composition comprising (2S,3R)-5,7-dihydroxy-2-(3,4,5-trihydroxyphenyl)chroman-3-yl 3,4,5-trihydroxybenzoate or a pharmaceutically acceptable salt thereof.

A method of improving cognitive and behavioral impairment in a DS patient in need thereof comprising administering to said patient a therapeutically effective amount of a pharmaceutically acceptable composition comprising (2R,3S)-5,7-dihydroxy-2-(3,4,5-trihydroxyphenyl)chroman-3-yl 3,4,5-trihydroxybenzoate or a pharmaceutically acceptable salt thereof.

A method of improving cognitive and behavioral impairment in a DS patient in need thereof comprising administering to said patient a therapeutically effective amount of a pharmaceutically acceptable composition comprising (2S,3R)-2-(3,4-dihydroxyphenyl)-5,7-dihydroxychroman-3-yl 3,4,5-trihydroxybenzoate or a pharmaceutically acceptable salt thereof.

A method of improving cognitive and behavioral impairment in a DS patient in need thereof comprising administering to said patient a therapeutically effective amount of a pharmaceutically acceptable composition comprising (2R,3S)-2-(3,4-dihydroxyphenyl)-5,7-dihydroxychroman-3-yl 3,4,5-trihydroxybenzoate or a pharmaceutically acceptable salt thereof.

A method of improving cognitive and behavioral impairment in a DS patient in need thereof comprising administering to said patient a therapeutically effective amount of a pharmaceutically acceptable composition comprising (2R,3R)-2-(3,4-dihydroxyphenyl)-5,7-dihydroxychroman-3-yl 3,4,5-trihydroxybenzoate or a pharmaceutically acceptable salt thereof.

A method of improving cognitive and behavioral impairment in a DS patient in need thereof comprising administering to said patient a therapeutically effective amount of a pharmaceutically acceptable composition comprising (2S,3S)-2-(3,4-dihydroxyphenyl)-5,7-dihydroxychroman-3-yl 3,4,5-trihydroxybenzoate or a pharmaceutically acceptable salt thereof.

A method of improving cognitive and behavioral impairment in a DS patient having AD in need thereof comprising administering to said patient a therapeutically effective amount of a pharmaceutically acceptable composition comprising a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate, (2R,3S)-2-(3,4-dihydroxyphenyl)-5,7-dihydroxychroman-3-yl 3,4,5-trihydroxybenzoate, (2R,3R)-2-(3,4-dihydroxyphenyl)-5,7-dihydroxychroman-3-yl 3,4,5-trihydroxybenzoate and (2S,3S)-2-(3,4-dihydroxyphenyl)-5,7-dihydroxychroman-3-yl 3,4,5-trihydroxybenzoate or a pharmaceutically acceptable salt thereof.

A method of improving cognitive and behavioral impairment in a DS patient having AD in need thereof comprising administering to said patient a therapeutically effective amount of a pharmaceutically acceptable composition comprising a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate and (2R,3S)-2-(3,4-dihydroxyphenyl)-5,7-dihydroxychroman-3-yl 3,4,5-trihydroxybenzoate or a pharmaceutically acceptable salt thereof.

A method of improving cognitive and behavioral impairment in a DS patient having AD in need thereof comprising administering to said patient a therapeutically effective amount of a pharmaceutically acceptable composition comprising (2S,3R)-5,7-dihydroxy-2-(3,4,5-trihydroxyphenyl)chroman-3-yl 3,4,5-trihydroxybenzoate or a pharmaceutically acceptable salt thereof.

A method of improving cognitive and behavioral impairment in a DS patient having AD in need thereof comprising administering to said patient a therapeutically effective amount of a pharmaceutically acceptable composition comprising (2R,3S)-5,7-dihydroxy-2-(3,4,5-trihydroxyphenyl)chroman-3-yl 3,4,5-trihydroxybenzoate or a pharmaceutically acceptable salt thereof.

A method of improving cognitive and behavioral impairment in a DS patient having AD in need thereof comprising administering to said patient a therapeutically effective amount of a pharmaceutically acceptable composition comprising (2S,3R)-2-(3,4-dihydroxyphenyl)-5,7-dihydroxychroman-3-yl 3,4,5-trihydroxybenzoate or a pharmaceutically acceptable salt thereof.

A method of improving cognitive and behavioral impairment in a DS patient having AD in need thereof comprising administering to said patient a therapeutically effective amount of a pharmaceutically acceptable composition comprising (2R,3S)-2-(3,4-dihydroxyphenyl)-5,7-dihydroxychroman-3-yl 3,4,5-trihydroxybenzoate or a pharmaceutically acceptable salt thereof.

A method of improving cognitive and behavioral impairment in a DS patient having AD in need thereof comprising administering to said patient a therapeutically effective amount of a pharmaceutically acceptable composition comprising (2R,3R)-2-(3,4-dihydroxyphenyl)-5,7-dihydroxychroman-3-yl 3,4,5-trihydroxybenzoate or a pharmaceutically acceptable salt thereof.

A method of improving cognitive and behavioral impairment in a DS patient having AD in need thereof comprising administering to said patient a therapeutically effective amount of a pharmaceutically acceptable composition comprising (2S,3S)-2-(3,4-dihydroxyphenyl)-5,7-dihydroxychroman-3-yl 3,4,5-trihydroxybenzoate or a pharmaceutically acceptable salt thereof.

A method of improving cognitive and behavioral impairment in an AD patient in need thereof comprising administering to said patient a therapeutically effective amount of a pharmaceutically acceptable composition comprising a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate, (2R,3S)-2-(3,4-dihydroxyphenyl)-5,7-dihydroxychroman-3-yl 3,4,5-trihydroxybenzoate, (2R,3R)-2-(3,4-dihydroxyphenyl)-5,7-dihydroxychroman-3-yl 3,4,5-trihydroxybenzoate and (2S,3S)-2-(3,4-dihydroxyphenyl)-5,7-dihydroxychroman-3-yl 3,4,5-trihydroxybenzoate or a pharmaceutically acceptable salt thereof.

A method of improving cognitive and behavioral impairment in an AD patient in need thereof comprising administering to said patient a therapeutically effective amount of a pharmaceutically acceptable composition comprising a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate and (2R,3S)-2-(3,4-dihydroxyphenyl)-5,7-dihydroxychroman-3-yl 3,4,5-trihydroxybenzoate or a pharmaceutically acceptable salt thereof.

A method of improving cognitive and behavioral impairment in an AD patient in need thereof comprising administering to said patient a therapeutically effective amount of a pharmaceutically acceptable composition comprising (2S,3R)-5,7-dihydroxy-2-(3,4,5-trihydroxyphenyl)chroman-3-yl 3,4,5-trihydroxybenzoate or a pharmaceutically acceptable salt thereof.

A method of improving cognitive and behavioral impairment in an AD patient in need thereof comprising administering to said patient a therapeutically effective amount of a pharmaceutically acceptable composition comprising (2R,3S)-5,7-dihydroxy-2-(3,4,5-trihydroxyphenyl)chroman-3-yl 3,4,5-trihydroxybenzoate or a pharmaceutically acceptable salt thereof.

A method of improving cognitive and behavioral impairment in an AD patient in need thereof comprising administering to said patient a therapeutically effective amount of a pharmaceutically acceptable composition comprising (2S,3R)-2-(3,4-dihydroxyphenyl)-5,7-dihydroxychroman-3-yl 3,4,5-trihydroxybenzoate or a pharmaceutically acceptable salt thereof.

A method of improving cognitive and behavioral impairment in an AD patient in need thereof comprising administering to said patient a therapeutically effective amount of a pharmaceutically acceptable composition comprising (2R,3S)-2-(3,4-dihydroxyphenyl)-5,7-dihydroxychroman-3-yl 3,4,5-trihydroxybenzoate or a pharmaceutically acceptable salt thereof.

A method of improving cognitive and behavioral impairment in an AD patient in need thereof comprising administering to said patient a therapeutically effective amount of a pharmaceutically acceptable composition comprising (2R,3R)-2-(3,4-dihydroxyphenyl)-5,7-dihydroxychroman-3-yl 3,4,5-trihydroxybenzoate or a pharmaceutically acceptable salt thereof.

A method of improving cognitive and behavioral impairment in an AD patient in need thereof comprising administering to said patient a therapeutically effective amount of a pharmaceutically acceptable composition comprising (2S,3S)-2-(3,4-dihydroxyphenyl)-5,7-dihydroxychroman-3-yl 3,4,5-trihydroxybenzoate or a pharmaceutically acceptable salt thereof.

A method of treating a neurodegenerative disease in a patient in need thereof comprising administering to said patient a therapeutically effective amount of a pharmaceutically acceptable composition comprising a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate, (2R,3S)-2-(3,4-dihydroxyphenyl)-5,7-dihydroxychroman-3-yl 3,4,5-trihydroxybenzoate, (2R,3R)-2-(3,4-dihydroxyphenyl)-5,7-dihydroxychroman-3-yl 3,4,5-trihydroxybenzoate and (2S,3S)-2-(3,4-dihydroxyphenyl)-5,7-dihydroxychroman-3-yl 3,4,5-trihydroxybenzoate or a pharmaceutically acceptable salt thereof.

A method of treating a neurodegenerative disease in a patient in need thereof comprising administering to said patient a therapeutically effective amount of a pharmaceutically acceptable composition comprising a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate and (2R,3S)-2-(3,4-dihydroxyphenyl)-5,7-dihydroxychroman-3-yl 3,4,5-trihydroxybenzoate or a pharmaceutically acceptable salt thereof.

A method of treating a neurodegenerative disease in a patient in need thereof comprising administering to said patient a therapeutically effective amount of a pharmaceutically acceptable composition comprising (2S,3R)-5,7-dihydroxy-2-(3,4,5-trihydroxyphenyl)chroman-3-yl 3,4,5-trihydroxybenzoate or a pharmaceutically acceptable salt thereof.

A method of treating a neurodegenerative disease in a patient in need thereof comprising administering to said patient a therapeutically effective amount of a pharmaceutically acceptable composition comprising (2R,3S)-5,7-dihydroxy-2-(3,4,5-trihydroxyphenyl)chroman-3-yl 3,4,5-trihydroxybenzoate or a pharmaceutically acceptable salt thereof.

A method of treating a neurodegenerative disease in a patient in need thereof comprising administering to said patient a therapeutically effective amount of a pharmaceutically acceptable composition comprising (2S,3R)-2-(3,4-dihydroxyphenyl)-5,7-dihydroxychroman-3-yl 3,4,5-trihydroxybenzoate or a pharmaceutically acceptable salt thereof.

A method of treating a neurodegenerative disease in a patient in need thereof comprising administering to said patient a therapeutically effective amount of a pharmaceutically acceptable composition comprising (2R,3S)-5,7-dihydroxy-2-(3,4,5-trihydroxyphenyl)chroman-3-yl 3,4,5-trihydroxybenzoate or a pharmaceutically acceptable salt thereof.

A method of treating a neurodegenerative disease in a patient in need thereof comprising administering to said patient a therapeutically effective amount of a pharmaceutically acceptable composition comprising (2R,3R)-2-(3,4-dihydroxyphenyl)-5,7-dihydroxychroman-3-yl 3,4,5-trihydroxybenzoate or a pharmaceutically acceptable salt thereof.

A method of treating a neurodegenerative disease in a patient in need thereof comprising administering to said patient a therapeutically effective amount of a pharmaceutically acceptable composition comprising (2S,3S)-2-(3,4-dihydroxyphenyl)-5,7-dihydroxychroman-3-yl 3,4,5-trihydroxybenzoate or a pharmaceutically acceptable salt thereof.

A method of treating cancer in a patient in need thereof comprising administering to said patient a therapeutically effective amount of a pharmaceutically acceptable composition comprising a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate, (2R,3S)-2-(3,4-dihydroxyphenyl)-5,7-dihydroxychroman-3-yl 3,4,5-trihydroxybenzoate, (2R,3R)-2-(3,4-dihydroxyphenyl)-5,7-dihydroxychroman-3-yl 3,4,5-trihydroxybenzoate and (2S,3S)-2-(3,4-dihydroxyphenyl)-5,7-dihydroxychroman-3-yl 3,4,5-trihydroxybenzoate or a pharmaceutically acceptable salt thereof.

A method of treating cancer in a patient in need thereof comprising administering to said patient a therapeutically effective amount of a pharmaceutically acceptable composition comprising a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-tri hydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate and (2R,3S)-2-(3,4-dihydroxyphenyl)-5,7-dihydroxychroman-3-yl 3,4,5-trihydroxybenzoate or a pharmaceutically acceptable salt thereof.

A method of treating cancer in a patient in need thereof comprising administering to said patient a therapeutically effective amount of a pharmaceutically acceptable composition comprising (2S,3R)-5,7-dihydroxy-2-(3,4,5-trihydroxyphenyl)chroman-3-yl 3,4,5-trihydroxybenzoate or a pharmaceutically acceptable salt thereof.

A method of treating cancer in a patient in need thereof comprising administering to said patient a therapeutically effective amount of a pharmaceutically acceptable composition comprising (2R,3S)-5,7-dihydroxy-2-(3,4,5-trihydroxyphenyl)chroman-3-yl 3,4,5-trihydroxybenzoate or a pharmaceutically acceptable salt thereof.

A method of treating cancer in a patient in need thereof comprising administering to said patient a therapeutically effective amount of a pharmaceutically acceptable composition comprising (2S,3R)-2-(3,4-dihydroxyphenyl)-5,7-dihydroxychroman-3-yl 3,4,5-trihydroxybenzoate or a pharmaceutically acceptable salt thereof.

A method of treating cancer in a patient in need thereof comprising administering to said patient a therapeutically effective amount of a pharmaceutically acceptable composition comprising (2R,3S)-5,7-dihydroxy-2-(3,4,5-trihydroxyphenyl)chroman-3-yl 3,4,5-trihydroxybenzoate or a pharmaceutically acceptable salt thereof.

A method of treating cancer in a patient in need thereof comprising administering to said patient a therapeutically effective amount of a pharmaceutically acceptable composition comprising (2R,3R)-2-(3,4-dihydroxyphenyl)-5,7-dihydroxychroman-3-yl 3,4,5-trihydroxybenzoate or a pharmaceutically acceptable salt thereof.

A method of treating cancer in a patient in need thereof comprising administering to said patient a therapeutically effective amount of a pharmaceutically acceptable composition comprising (2S,3S)-2-(3,4-dihydroxyphenyl)-5,7-dihydroxychroman-3-yl 3,4,5-trihydroxybenzoate or a pharmaceutically acceptable salt thereof.

A method of treating a DYRK1A-mediated disorder in a patient in need thereof comprising administering to said patient a therapeutically effective amount of a pharmaceutically acceptable composition comprising a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate, (2R,3S)-2-(3,4-dihydroxyphenyl)-5,7-dihydroxychroman-3-yl 3,4,5-trihydroxybenzoate, (2R,3R)-2-(3,4-dihydroxyphenyl)-5,7-dihydroxychroman-3-yl 3,4,5-trihydroxybenzoate and (2S,3S)-2-(3,4-dihydroxyphenyl)-5,7-dihydroxychroman-3-yl 3,4,5-trihydroxybenzoate or a pharmaceutically acceptable salt thereof.

A method of treating a DYRK1A-mediated disorder in a patient in need thereof comprising administering to said patient a therapeutically effective amount of a pharmaceutically acceptable composition comprising a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate and (2R,3S)-2-(3,4-dihydroxyphenyl)-5,7-dihydroxychroman-3-yl 3,4,5-trihydroxybenzoate or a pharmaceutically acceptable salt thereof.

A method of treating a DYRK1A-mediated disorder in a patient in need thereof comprising administering to said patient a therapeutically effective amount of a pharmaceutically acceptable composition comprising (2S,3R)-5,7-dihydroxy-2-(3,4,5-trihydroxyphenyl)chroman-3-yl 3,4,5-trihydroxybenzoate or a pharmaceutically acceptable salt thereof.

A method of treating a DYRK1A-mediated disorder in a patient in need thereof comprising administering to said patient a therapeutically effective amount of a pharmaceutically acceptable composition comprising (2R,3S)-5,7-dihydroxy-2-(3,4,5-trihydroxyphenyl)chroman-3-yl 3,4,5-trihydroxybenzoate or a pharmaceutically acceptable salt thereof.

A method of treating a DYRK1A-mediated disorder in a patient in need thereof comprising administering to said patient a therapeutically effective amount of a pharmaceutically acceptable composition comprising (2S,3R)-2-(3,4-dihydroxyphenyl)-5,7-dihydroxychroman-3-yl 3,4,5-trihydroxybenzoate or a pharmaceutically acceptable salt thereof.

A method of treating a DYRK1A-mediated disorder in a patient in need thereof comprising administering to said patient a therapeutically effective amount of a pharmaceutically acceptable composition comprising (2R,3S)-2-(3,4-dihydroxyphenyl)-5,7-dihydroxychroman-3-yl 3,4,5-trihydroxybenzoate or a pharmaceutically acceptable salt thereof.

A method of treating a DYRK1A-mediated disorder in a patient in need thereof comprising administering to said patient a therapeutically effective amount of a pharmaceutically acceptable composition comprising (2R,3R)-2-(3,4-dihydroxyphenyl)-5,7-dihydroxychroman-3-yl 3,4,5-trihydroxybenzoate or a pharmaceutically acceptable salt thereof.

A method of treating a DYRK1A-mediated disorder in a patient in need thereof comprising administering to said patient a therapeutically effective amount of a pharmaceutically acceptable composition comprising (2S,3S)-2-(3,4-dihydroxyphenyl)-5,7-dihydroxychroman-3-yl 3,4,5-trihydroxybenzoate or a pharmaceutically acceptable salt thereof.

A method of modulating DYRK1A comprising contacting DYRK1A with a DYRK1A-modulating amount of a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate, (2R,3S)-2-(3,4-dihydroxyphenyl)-5,7-dihydroxychroman-3-yl 3,4,5-trihydroxybenzoate, (2R,3R)-2-(3,4-dihydroxyphenyl)-5,7-dihydroxychroman-3-yl 3,4,5-trihydroxybenzoate and (2S,3S)-2-(3,4-dihydroxyphenyl)-5,7-dihydroxychroman-3-yl 3,4,5-trihydroxybenzoate or a pharmaceutically acceptable salt thereof.

A method of modulating DYRK1A comprising contacting DYRK1A with a DYRK1A-modulating amount of a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate and (2R,3S)-2-(3,4-dihydroxyphenyl)-5,7-dihydroxychroman-3-yl 3,4,5-trihydroxybenzoate or a pharmaceutically acceptable salt thereof.

A method of modulating DYRK1A comprising contacting DYRK1A with a DYRK1A-modulating amount of (2S,3R)-5,7-dihydroxy-2-(3,4,5-trihydroxyphenyl)chroman-3-yl 3,4,5-trihydroxybenzoate or a pharmaceutically acceptable salt thereof.

A method of modulating DYRK1A comprising contacting DYRK1A with a DYRK1A-modulating amount of (2R,3S)-5,7-dihydroxy-2-(3,4,5-trihydroxyphenyl)chroman-3-yl 3,4,5-trihydroxybenzoate or a pharmaceutically acceptable salt thereof.

A method of modulating DYRK1A comprising contacting DYRK1A with a DYRK1A-modulating amount (2S,3R)-2-(3,4-dihydroxyphenyl)-5,7-dihydroxy chroman-3-yl 3,4,5-trihydroxybenzoate or a pharmaceutically acceptable salt thereof.

A method of modulating DYRK1A comprising contacting DYRK1A with a DYRK1A-modulating amount (2R,3S)-2-(3,4-dihydroxyphenyl)-5,7-dihydroxy chroman-3-yl 3,4,5-trihydroxybenzoate or a pharmaceutically acceptable salt thereof.

A method of modulating DYRK1A comprising contacting DYRK1A with a DYRK1A-modulating amount of (2R,3R)-2-(3,4-dihydroxyphenyl)-5,7-dihydroxy chroman-3-yl 3,4,5-trihydroxybenzoate or a pharmaceutically acceptable salt thereof.

A method of modulating DYRK1A comprising contacting DYRK1A with a DYRK1A-modulating amount of (2S,3S)-2-(3,4-dihydroxyphenyl)-5,7-dihydroxychroman-3-yl 3,4,5-trihydroxybenzoate or a pharmaceutically acceptable salt thereof.

A method of inhibiting DYRK1A comprising contacting DYRK1A with a DYRK1A-inhibiting amount of a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate, (2R,3S)-2-(3,4-dihydroxyphenyl)-5,7-dihydroxychroman-3-yl 3,4,5-trihydroxybenzoate, (2R,3R)-2-(3,4-dihydroxyphenyl)-5,7-dihydroxychroman-3-yl 3,4,5-trihydroxybenzoate and (2S,3S)-2-(3,4-dihydroxyphenyl)-5,7-dihydroxychroman-3-yl 3,4,5-trihydroxybenzoate or a pharmaceutically acceptable salt thereof.

A method of inhibiting DYRK1A comprising contacting DYRK1A with a DYRK1A-inhibiting amount of a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate and (2R,3S)-2-(3,4-dihydroxyphenyl)-5,7-dihydroxychroman-3-yl 3,4,5-trihydroxybenzoate or a pharmaceutically acceptable salt thereof.

A method of inhibiting DYRK1A comprising contacting DYRK1A with a DYRK1A-inhibiting amount of (2S,3R)-5,7-dihydroxy-2-(3,4,5-trihydroxyphenyl)chroman-3-yl 3,4,5-trihydroxybenzoate or a pharmaceutically acceptable salt thereof.

A method of inhibiting DYRK1A comprising contacting DYRK1A with a DYRK1A-inhibiting amount of (2R,3S)-5,7-dihydroxy-2-(3,4,5-trihydroxyphenyl)chroman-3-yl 3,4,5-trihydroxybenzoate or a pharmaceutically acceptable salt thereof.

A method of inhibiting DYRK1A comprising contacting DYRK1A with a DYRK1A-inhibiting amount (2S,3R)-2-(3,4-dihydroxyphenyl)-5,7-dihydroxychroman-3-yl 3,4,5-trihydroxybenzoate or a pharmaceutically acceptable salt thereof.

A method of inhibiting DYRK1A comprising contacting DYRK1A with a DYRK1A-inhibiting amount (2R,3S)-2-(3,4-dihydroxyphenyl)-5,7-dihydroxychroman-3-yl 3,4,5-trihydroxybenzoate or a pharmaceutically acceptable salt thereof.

A method of inhibiting DYRK1A comprising contacting DYRK1A with a DYRK1A-inhibiting amount of (2R,3R)-2-(3,4-dihydroxyphenyl)-5,7-dihydroxychroman-3-yl 3,4,5-trihydroxybenzoate or a pharmaceutically acceptable salt thereof.

A method of inhibiting DYRK1A comprising contacting DYRK1A with a DYRK1A-inhibiting amount of (2S,3S)-2-(3,4-dihydroxyphenyl)-5,7-dihydroxychroman-3-yl 3,4,5-trihydroxybenzoate or a pharmaceutically acceptable salt thereof.

### DETAILED DESCRIPTION OF THE INVENTION

Various definitions are provided herein, explicitly and/or through usage, and it is understood that such definitions will be applied by those of skill in the art in understanding the present invention.

The terms “a” and “an” do not denote a limitation of quantity, but rather denote the presence of at least one of the referenced item.

The term “patient” includes all mammals, e.g., non-human mammals and humans.

The term “cognitive and behavioral impairment” includes but is not limited to short attention span, mild cognitive impairment, memory impairment, poor judgment, slow learning, delayed language and speech development, mental retardation, general anxiety, depression, repetitive and obsessive-compulsive behaviors, oppositional, impulsive, hyperactive and inattentive behaviors, sleep related difficulties, autism spectrum conditions, deficits in social relatedness and self-immersed behaviors, and any subset of combinations thereof.

The nomenclature and structural formulas of catechins as used herein are provided below in

TABLE 1
Catechin Nomenclature and Structures
Catechin (C)

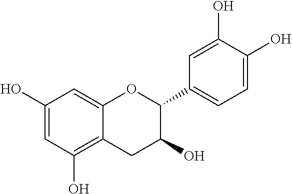 [SMILES: Oc1cc(O)c2c(c1)O[C@H](c1ccc(O)c(O)c1)[C@@H](O)C2]

(2R,3S)-2-(3,4-dihydroxyphenyl)chroman-3,5,7-triol

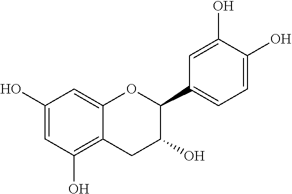 [SMILES: Oc1cc(O)c2c(c1)O[C@@H](c1ccc(O)c(O)c1)[C@H](O)C2]

(2S,3R)-2-(3,4-dihydroxyphenyl)chroman-3,5,7-triol
Gallocatechin (GC)

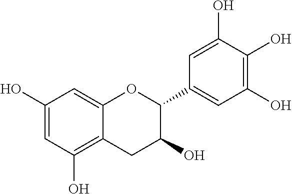 [SMILES: Oc1cc(O)c2c(c1)O[C@H](c1cc(O)c(O)c(O)c1)[C@@H](O)C2]

(2R,3S)-2-(3,4,5-trihydroxyphenyl)chroman-3,5,7-triol

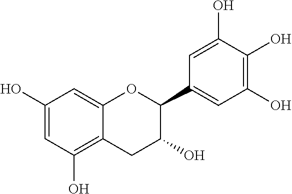 [SMILES: Oc1cc(O)c2c(c1)O[C@@H](c1cc(O)c(O)c(O)c1)[C@H](O)C2]

(2S,3R)-2-(3,4,5-trihydroxyphenyl)chroman-3,5,7-triol
Epicatechin (EC)

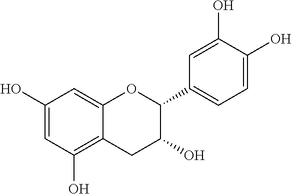 [SMILES: Oc1cc(O)c2c(c1)O[C@H](c1ccc(O)c(O)c1)[C@H](O)C2]

(2R,3R)-2-(3,4-dihydroxyphenyl)chroman-3,5,7-triol

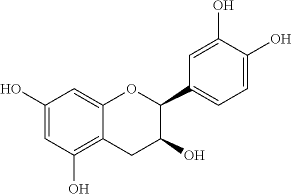 [SMILES: Oc1cc(O)c2c(c1)O[C@@H](c1ccc(O)c(O)c1)[C@@H](O)C2]

(2S,3S)-2-(3,4-dihydroxyphenyl)chroman-3,5,7-triol
Epigallocatechin (EGC)

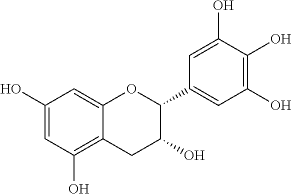 [SMILES: Oc1cc(O)c2c(c1)O[C@H](c1cc(O)c(O)c(O)c1)[C@H](O)C2]

(2R,3R)-2-(3,4,5-trihydroxyphenyl)chroman-3,5,7-
triol

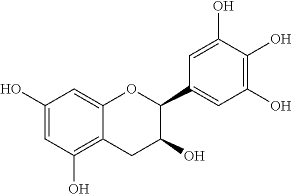 [SMILES: Oc1cc(O)c2c(c1)O[C@@H](c1cc(O)c(O)c(O)c1)[C@@H](O)C2]

(2S,3S)-2-(3,4,5-trihydroxyphenyl)chroman-3,5,7-
triol
Epigallocatechin Gallate (EGCG)

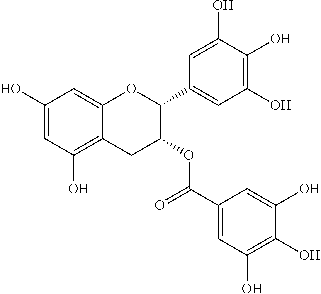 [SMILES: O=C(O[C@@H]1Cc2c(O)cc(O)cc2O[C@@H]1c1cc(O)c(O)c(O)c1)c1cc(O)c(O)c(O)c1]

(2R,3R)-5,7-dihydroxy-2-(3,4,5-trihydroxyphenyl)chroman-3-yl 3,4,5-trihydroxybenzoate

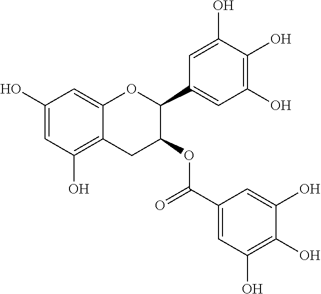 [SMILES: O=C(O[C@H]1Cc2c(O)cc(O)cc2O[C@H]1c1cc(O)c(O)c(O)c1)c1cc(O)c(O)c(O)c1]

(2S,3S)-5,7-dihydroxy-2-(3,4,5-trihydroxyphenyl)chroman-3-yl 3,4,5-trihydroxybenzoate
Epigallocatechin Gallate O-methyl Metabolites

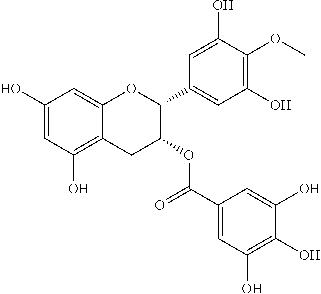 [SMILES: COc1c(O)cc([C@H]2Oc3cc(O)cc(O)c3C[C@H]2OC(=O)c2cc(O)c(O)c(O)c2)cc1O]

(2R,3R)-2-(3,5-dihydroxy-4-methoxyphenyl)-5,7-dihydroxychroman-3-yl 3,4,5-trihydroxybenzoate

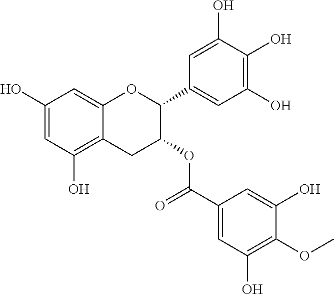 [SMILES: COc1c(O)cc(C(=O)O[C@@H]2Cc3c(O)cc(O)cc3O[C@@H]2c2cc(O)c(O)c(O)c2)cc1O]

(2R,3R)-5,7-dihydroxy-2-(3,4,5-trihydroxyphenyl)chroman-3-yl 3,5-dihydroxy-4-methoxybenzoate
Gallocatechin Gallate (GCG)

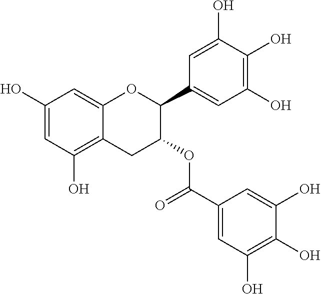 [SMILES: O=C(O[C@@H]1Cc2c(O)cc(O)cc2O[C@H]1c1cc(O)c(O)c(O)c1)c1cc(O)c(O)c(O)c1]

(2S,3R)-5,7-dihydroxy-2-(3,4,5-trihydroxyphenyl)chroman-3-yl 3,4,5-trihydroxybenzoate

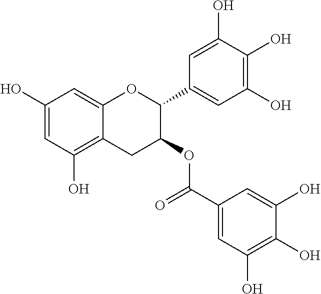 [SMILES: O=C(O[C@H]1Cc2c(O)cc(O)cc2O[C@@H]1c1cc(O)c(O)c(O)c1)c1cc(O)c(O)c(O)c1]

(2R,3S)-5,7-dihydroxy-2-(3,4,5-trihydroxyphenyl)chroman-3-yl 3,4,5-trihydroxybenzoate
Catechin Gallate (CG)

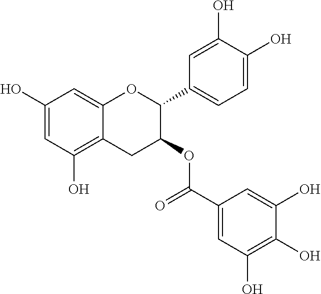 [SMILES: O=C(O[C@H]1Cc2c(O)cc(O)cc2O[C@@H]1c1ccc(O)c(O)c1)c1cc(O)c(O)c(O)c1]

(2R,3S)-2-(3,4-dihydroxyphenyl)-5,7-dihydroxychroman-3-yl 3,4,5-
trihydroxybenzoate

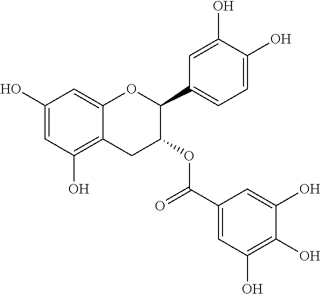 [SMILES: O=C(O[C@@H]1Cc2c(O)cc(O)cc2O[C@H]1c1ccc(O)c(O)c1)c1cc(O)c(O)c(O)c1]

(2S,3R)-2-(3,4-dihydroxyphenyl)-5,7-dihydroxychroman-3-yl 3,4,5-
trihydroxybenzoate
Epicatechin Gallate (ECG)

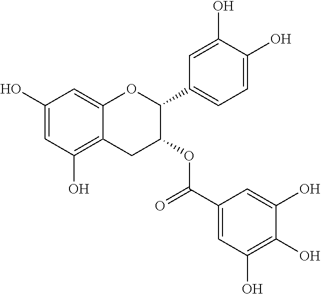 [SMILES: O=C(O[C@@H]1Cc2c(O)cc(O)cc2O[C@@H]1c1ccc(O)c(O)c1)c1cc(O)c(O)c(O)c1]

(2R,3R)-2-(3,4-dihydroxyphenyl)-5,7-dihydroxychroman-3-yl 3,4,5-
trihydroxybenzoate

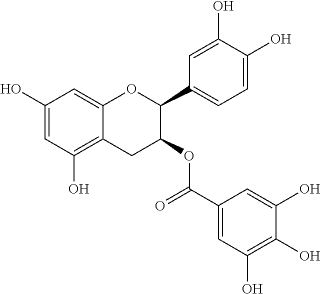 [SMILES: O=C(O[C@H]1Cc2c(O)cc(O)cc2O[C@H]1c1ccc(O)c(O)c1)c1cc(O)c(O)c(O)c1]

(2S,3S)-2-(3,4-dihydroxyphenyl)-5,7-dihydroxychroman-3-yl 3,4,5-
trihydroxybenzoate

It is to be understood that the present invention includes any and all possible pharmaceutically acceptable salts, solvates (including hydrates) of the free compound or salt, crystalline and non-crystalline forms, as well as various polymorphs of the compound.

The term “pharmaceutically acceptable composition” includes any suitable formulation including, for example, capsules, tablets, injections and liquids and may be administered through any suitable route including oral, buccal, parenteral, intravenous, intramuscular, rectal, transdermal and the like. Excipients used to formulate the pharmaceutical formulations may be any of those suitable for the respective dosage form such as fillers, stabilizers, extenders, binders, humidifiers, surfactants, lubricants, and the like.

The term “therapeutically effective amount” means an amount effective, when administered to a patient, to provide any therapeutic benefit. A therapeutic benefit may be an amelioration of symptoms, e.g., an amount effective to decrease the symptoms. In certain circumstances a patient may not present symptoms of a condition for which the patient is being treated. Thus a therapeutically effective amount of a compound is also an amount sufficient to provide a significant positive effect on any indicia of a disease, disorder or condition.

Frequency of dosage may vary depending on the active agent used and the particular disorder being treated. For most disorders a dosage regimen of once or twice per day is suitable. It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease in the patient undergoing therapy. Patients may generally be monitored for therapeutic effectiveness using assays suitable for the condition being treated or prevented, which will be familiar to those of ordinary skill in the art.

The term “contacting DYRK1A with a DYRK1A-modulating amount of a compound” includes providing in any manner said compound to permit contact, binding or any other interaction with DYRK1A whether administered in vivo, ex vivo or in vitro.

Various references are cited throughout the Specification, each of which is incorporated herein by reference in its entirety. The citation of references herein shall not be construed as an admission that such is prior art to the present invention.

Other embodiments of the present invention may comprise a suitable combination of two or more of the embodiments and/or aspects disclosed herein.

The present invention may be embodied in other forms or carried out in other ways without departing from the spirit or essential characteristics thereof. The present disclosure is therefore to be considered as in all aspects illustrated and not restrictive, the scope of the invention being indicated by the appended Claims, and all changes which come within the meaning and range of equivalency are intended to be embraced therein.

The discussion herein and the following examples set forth and illustrate various exemplary embodiments of the present invention, which are understood to be illustrative and non-limiting.

### Example 1. DYRK1 IC50's

Table 2. DYRK1A Inhibition by Catechins in Example 1 provides the inhibitory concentrations of various catechins on DYRK1A.

TABLE 2
DYRK1A Inhibition by Catechins

| | DYRK1A IC₅₀ |
|---|---|
| Catechin | (μM) |
| (2S,3R)-2-(3,4,5-trihydroxyphenyl)chroman-3,5,7-triol | >100 |
| (2R,3R)-2-(3,4,5-trihydroxyphenyl)chroman-3,5,7-triol | >100 |
| (2S,3R)-2-(3,4-dihydroxyphenyl)chroman-3,5,7-triol | >100 |
| (2R,3R)-2-(3,4-dihydroxyphenyl)chroman-3,5,7-triol | >100 |
| (2R,3R)-5,7-dihydroxy-2-(3,4,5-trihydroxyphenyl)chroman-3-yl 3,4,5- | 0.22 |
| trihydroxybenzoate | |
| (2S,3R)-5,7-dihydroxy-2-(3,4,5-trihydroxyphenyl)chroman-3-yl 3,4,5- | 0.15 |
| trihydroxybenzoate | |
| (2R,3R)-2-(3,4-dihydroxyphenyl)-5,7-dihydroxychroman-3-yl 3,4,5- | 0.52 |
| trihydroxybenzoate | |
| (2S,3R)-2-(3,4-dihydroxyphenyl)-5,7-dihydroxychroman-3-yl 3,4,5- | 0.18 |
| trihydroxybenzoate | |
| (2R,3R)-5,7-dihydroxy-2-(3,4,5-trihydroxyphenyl)chroman-3-yl 3,5- | 25.1 |
| dihydroxy-4-methoxybenzoate | |
| (2R,3R)-2-(3,5-dihydroxy-4-methoxyphenyl)-5,7-dihydroxychroman-3-yl | 5.8 |
| 3,4,5-trihydroxybenzoate | |

### Example 2. DYRK1A ELISA Assay

The results in Table 2 resulted from performing the following assay as described in Liu Y, Adayev T and Hwang Y W. An ELISA DYRK1A non-radioactive kinase assay suitable for the characterization of inhibitors [version 2; referees: 2 approved] F1000Research 2017, 6:42 (doi: 10.12688/f1000research.10582.2) and summarized below:

Coating.

Dilute HT-PRD (6× histidine tagged dynamin 1a proline-rich domain, 118 residues plus 6 histidines and 1 methionine) in Tris dilution buffer (25 mM Tris-HCl, pH 7.4 and 100 mM NaCl) to the concentration of 2 ng/μ1 and coat each well (96-well plate) with 100 μl diluted HT-PRD (200 ng total) for overnight at 4° C.

Blocking.

Decant HT-PRD solution, wash the wells twice with Tris dilution buffer. Block each well with 150 μl BSA/PBST blocking buffer PBST (2% BSA in PBS with 0.25% Tween 20) for at least 1 hr at room temperature.

Phosphorylation.

Decant the blocking solution and wash the wells 3-4 times with Tris dilution buffer. Perform DYRK1A phosphorylation in the well with 100 μl reaction buffer (25 mM HEPES, pH 7.4, 100 mM NaCl, 5 mM MgCl₂, and 100 μM ATP) containing DYRK1A (eg. 5-10 ng purified HT-DYRK1A) and inhibitor, if necessary, for 30 min at 30° C. Suggested protocol:
1. Prepare reaction mixture with 1.25× concentrated reaction buffer containing 125 μM ATP. Use 80 μl of this mixture in the reaction will give final ATP concentration of 100 μM. ATP concentrations may be adjusted if necessary.
2. Add 10 μl inhibitor. If the inhibitor is dissolved in DMSO, shake the plate gently for 1 min before adding kinase.
3. Dilute DYRK1A in Tris dilution buffer to a concentration of 0.5 ng/μl (prepare fresh every time).
4. Initiate phosphorylation by adding 10 μl DYRK1A to each well.

Primary Antibody.

Decant the phosphorylation solution and wash the wells three times with PBST. Add 100 μl diluted antibody 3D3 (eg. 2˜3K dilution of 1.5 mg/ml stock in BSA blocking buffer) and incubate for 1 hr at room temperature.

Secondary Antibody.

Decant the primary antibody solution and wash the wells three times with PBST. Add 100 μl diluted AP-conjugated anti-mouse IgG (5K dilution of anti-mouse IgG-AP in BSA blocking buffer) and incubate for 1 hr at room temperature.

Color Development and Detection.

Decant the secondary antibody and wash the wells extensively (3-4 times) with AP buffer (10 mM Tris-HCl, pH 9.5, 10 mM NaCl, and 5 mM MgCl₂). Add 100 μl p-nitrophenyl phosphate solution (1 mg/ml in AP buffer or 1 PNPP tablet per 5 ml 1× diethanolamine substrate buffer) and incubate the plate at 30° C. Yellow color should start appearing in a few minutes. OD at 405 nm usually can be read between 10-30 min.

What is claimed is:

1. A method of improving cognitive and behavioral impairment in an Alzheimer's disease patient in need thereof comprising administering to said patient a therapeutically effective amount of a pharmaceutically acceptable composition comprising a compound selected from the group consisting of
(2S,3R)-5,7-dihydroxy-2-(3,4,5-trihydroxyphenyl)chroman-3-yl 3,4,5-trihydroxybenzoate,
(2R,3S)-5,7-dihydroxy-2-(3,4,5-trihydroxyphenyl)chroman-3-yl 3,4,5-trihydroxybenzoate,
(2S,3R)-2-(3,4-dihydroxyphenyl)-5,7-dihydroxychroman-3-yl 3,4,5-trihydroxybenzoate,
(2R,3S)-2-(3,4-dihydroxyphenyl)-5,7-dihydroxychroman-3-yl 3,4,5-trihydroxybenzoate,
(2R,3R)-2-(3,4-dihydroxyphenyl)-5,7-dihydroxy chroman-3-yl 3,4,5-trihydroxybenzoate, and
(2S,3S)-2-(3,4-dihydroxyphenyl)-5,7-dihydroxychroman-3-yl 3,4,5-trihydroxybenzoate
or a pharmaceutically acceptable salt thereof.

2. The method according to claim 1, comprising a compound selected from the group consisting of
(2S,3R)-5,7-dihydroxy-2-(3,4,5-trihydroxyphenyl)chroman-3-yl 3,4,5-trihydroxybenzoate,
(2R,3S)-5,7-dihydroxy-2-(3,4,5-trihydroxyphenyl)chroman-3-yl 3,4,5-trihydroxybenzoate,
(2S,3R)-2-(3,4-dihydroxyphenyl)-5,7-dihydroxychroman-3-yl 3,4,5-trihydroxybenzoate, and
(2R,3S)-2-(3,4-dihydroxyphenyl)-5,7-dihydroxychroman-3-yl 3,4,5-trihydroxybenzoate or a pharmaceutically acceptable salt thereof.

3. The method according to claim 1, comprising (2S,3R)-5,7-dihydroxy-2-(3,4,5-trihydroxyphenyl)chroman-3-yl 3,4,5-trihydroxybenzoate or a pharmaceutically acceptable salt thereof.

4. The method according to claim 1, comprising (2R,3S)-5,7-dihydroxy-2-(3,4,5-trihydroxyphenyl)chroman-3-yl 3,4,5-trihydroxybenzoate or a pharmaceutically acceptable salt thereof.

5. The method according to claim 1, comprising (2S,3R)-2-(3,4-dihydroxyphenyl)-5,7-dihydroxychroman-3-yl 3,4,5-trihydroxybenzoate or a pharmaceutically acceptable salt thereof.

6. The method according to claim 1, comprising (2R,3S)-2-(3,4-dihydroxyphenyl)-5,7-dihydroxychroman-3-yl 3,4,5-trihydroxybenzoate or a pharmaceutically acceptable salt thereof.

7. The method according to claim 1, comprising (2R,3R)-2-(3,4-dihydroxyphenyl)-5,7-dihydroxychroman-3-yl 3,4,5-trihydroxybenzoate or a pharmaceutically acceptable salt thereof.

8. The method according to claim 1, comprising (2S,3S)-2-(3,4-dihydroxyphenyl)-5,7-dihydroxychroman-3-yl 3,4,5-trihydroxybenzoate or a pharmaceutically acceptable salt thereof.

9. A method of treating a DYRK1A-mediated disorder in a patient in need thereof comprising administering to said patient a therapeutically effective amount of a pharmaceutically acceptable composition comprising a compound selected from the group consisting of
(2S,3R)-5,7-dihydroxy-2-(3,4,5-trihydroxyphenyl)chroman-3-yl 3,4,5-trihydroxybenzoate,
(2R,3S)-5,7-dihydroxy-2-(3,4,5-trihydroxyphenyl)chroman-3-yl 3,4,5-trihydroxybenzoate,
(2S,3R)-2-(3,4-dihydroxyphenyl)-5,7-dihydroxychroman-3-yl 3,4,5-trihydroxybenzoate,
(2R,3S)-2-(3,4-dihydroxyphenyl)-5,7-dihydroxychroman-3-yl 3,4,5-trihydroxybenzoate,
(2R,3R)-2-(3,4-dihydroxyphenyl)-5,7-dihydroxy chroman-3-yl 3,4,5-trihydroxybenzoate, and
(2S,3S)-2-(3,4-dihydroxyphenyl)-5,7-dihydroxychroman-3-yl 3,4,5-trihydroxybenzoate
or a pharmaceutically acceptable salt thereof.

10. The method according to claim 9, comprising a compound selected from the group consisting of
(2S,3R)-5,7-dihydroxy-2-(3,4,5-trihydroxyphenyl)chroman-3-yl 3,4,5-trihydroxybenzoate,
(2R,3S)-5,7-dihydroxy-2-(3,4,5-trihydroxyphenyl)chroman-3-yl 3,4,5-trihydroxybenzoate,
(2S,3R)-2-(3,4-dihydroxyphenyl)-5,7-dihydroxychroman-3-yl 3,4,5-trihydroxybenzoate, and
(2R,3S)-2-(3,4-dihydroxyphenyl)-5,7-dihydroxychroman-3-yl 3,4,5-trihydroxybenzoate
or a pharmaceutically acceptable salt thereof.

11. The method according to claim 9, comprising (2S,3R)-5,7-dihydroxy-2-(3,4,5-trihydroxyphenyl)chroman-3-yl 3,4,5-trihydroxybenzoate or a pharmaceutically acceptable salt thereof.

12. The method according to claim 9, comprising (2R,3S)-5,7-dihydroxy-2-(3,4,5-trihydroxyphenyl)chroman-3-yl 3,4,5-trihydroxybenzoate or a pharmaceutically acceptable salt thereof.

13. The method according to claim 9, comprising (2S,3R)-2-(3,4-dihydroxyphenyl)-5,7-dihydroxychroman-3-yl 3,4,5-trihydroxybenzoate or a pharmaceutically acceptable salt thereof.

14. The method according to claim 9, comprising (2R,3S)-2-(3,4-dihydroxyphenyl)-5,7-dihydroxychroman-3-yl 3,4,5-trihydroxybenzoate or a pharmaceutically acceptable salt thereof.

15. The method according to claim 9, comprising (2R,3R)-2-(3,4-dihydroxyphenyl)-5,7-dihydroxychroman-3-yl 3,4,5-trihydroxybenzoate or a pharmaceutically acceptable salt thereof.

16. The method according to claim 9, comprising (2S,3S)-2-(3,4-dihydroxyphenyl)-5,7-dihydroxychroman-3-yl 3,4,5-trihydroxybenzoate or a pharmaceutically acceptable salt thereof.

17. A method of improving cognitive and behavioral impairment in a Down syndrome patient having Alzheimer's disease in need thereof comprising administering to said patient a therapeutically effective amount of a pharmaceutically acceptable composition comprising a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate, (2R,3S)-2-(3,4-dihydroxyphenyl)-5,7-dihydroxychroman-3-yl 3,4,5-trihydroxybenzoate, (2R,3R)-2-(3,4-dihydroxyphenyl)-5,7-dihydroxychroman-3-yl 3,4,5-trihydroxybenzoate and (2S,3S)-2-(3,4-dihydroxyphenyl)-5,7-dihydroxychroman-3-yl 3,4,5-trihydroxybenzoate or a pharmaceutically acceptable salt thereof.

18. The method according to claim 17 comprising a compound selected from the group consisting of (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2S,3R)-2-(3,4-dihydroxyphenyl)-5,7-dihydroxychroman-3-yl 3,4,5-trihydroxybenzoate and (2R,3S)-2-(3,4-dihydroxyphenyl)-5,7-dihydroxychroman-3-yl 3,4,5-trihydroxybenzoate or a pharmaceutically acceptable salt thereof.

19. The method according to claim 17 comprising administering to said patient a therapeutically effective amount of a pharmaceutically acceptable composition comprising (2S,3R)-5,7-dihydroxy-2-(3,4,5-trihydroxyphenyl)chroman-3-yl 3,4,5-trihydroxybenzoate or a pharmaceutically acceptable salt thereof.

20. The method according to claim 17 comprising administering to said patient a therapeutically effective amount of a pharmaceutically acceptable composition comprising (2R,3S)-5,7-dihydroxy-2-(3,4,5-trihydroxyphenyl)chroman-3-yl 3,4,5-trihydroxybenzoate or a pharmaceutically acceptable salt thereof.
